(19) United States
(12) Patent Application Publication
(10) Pub. No.: US 2026/0157962 A1
(43) Pub. Date: Jun. 11, 2026

(54) Compositions of Stannic Protoporfin for Long Term Stability and Container Closure Systems for Storing Stannic Protoporfin

(71) Applicant: Renibus Therapeutics, Inc., Southlake, TX (US)

(72) Inventor: Donald Jeffrey Keyser, Southlake, TX (US)

(21) Appl. No.: 19/413,414

(22) Filed: Dec. 9, 2025

Related U.S. Application Data
(60) Provisional application No. 63/729,911, filed on Dec. 9, 2024.

(51) Int. Cl. C07F 7/22; A61K 9/00; A61K 9/08; A61K 31/555

(52) U.S. Cl. CPC A61K 9/08; A61K 9/0019; A61K 31/555; C07F 7/2284; C07F 7/2296

(57) ABSTRACT

The present disclosure generally relates to novel stannic protoporfin products for long term stability including container closure systems for aqueous stannic protoporfin compositions that are adapted to preserve the composition in a stable form prior to use.

### TECHNICAL FIELD

The present disclosure generally relates to novel stannic protoporfin products for long term stability including container closure systems for aqueous stannic protoporfin compositions that are adapted to preserve the composition in a stable form prior to use.

### BACKGROUND

Stannic protoporfin (Sn-protoporphyrin or SnPP) is known agent that undergoes proximal tubule uptake where it activates redox sensitive transcription factors, leading to the up-regulation of redox sensitive cytoprotective proteins. However, known methods of making SnPP includes a complicated process involving four distinct intermediate compounds and is known to have problems with stability and purity.

SnPP is disclosed in U.S. Pat. No. 10,639,321 B2, entitled “Compositions, kits, and methods to induce acquired cytoresistance using stress protein inducers.” The '321 patent discloses that SnPP in combination with iron sucrose (FeS) can be used to induce acquired cytoresistance. SnPP is also known to have some antiviral effect for particular kinds of viruses as disclosed by Neris et al., “Co-protoporphyrin IX and Sn-protoporphyrin IX inactivate Zika, Chikungunya and other arboviruses by targeting the viral envelope,” Sci Rep. 2018 Jun. 28; 8(1):9805. doi: 10.1038/s41598-018-27855-7. Neris et al. disclose that SnPP can be photosensitized and that non-photosensitized versions of SnPP may be used in photodynamic therapy for microorganism killing.

The long term stability of SnPP suitable for use in pharmaceutical formulations has not been studied. Accordingly, novel SnPP products that are capable of long term storage are needed.

### BRIEF DESCRIPTION OF THE DRAWINGS

FIG. 1A shows the first step in the production of SnPP according to a 4-step process;

FIG. 1B shows the second step in the production of SnPP according to a 4-step process;

FIG. 1C shows the third step in the production of SnPP according to a 4-step process;

FIG. 1D shows the fourth step in the production of SnPP according to a 4-step process;

FIG. 2A shows the first step in the production of SnPP according to a two-step process;

FIG. 2B shows the second step in the production of SnPP according a two-step process;

FIG. 3 shows a comparison of the impurity profiles present in the product of a two-step process compared to that of the four-step process.

### DETAILED DESCRIPTION

The present inventors have discovered that aqueous SnPP compositions are susceptible to particulate formation over time. SnPP is known to be sensitive to heat and have photosensitivity issues, and may be susceptible to photodegradation. The present inventors have further discovered evidence that certain components in glass containers that are intended to shield the contents of the glass container from light may interact with the SnPP leading to particulate formation. In addition, when SnPP is manufactured to higher purity levels, the material appears to become more susceptible to agglomeration and particulate formation.

Tin (IV) protoporphyrin IX dichloride sterile solution (SnPP) is a high pH (˜12.4) solution formulated to contain 9 mg/mL of tin (IV) protoporphyrin dichloride, using the following batch composition:

TABLE 1
Batch Formula

| Component | Amount (per mL) |
|---|---|
| Tin(IV) protoporphyrin IX dichloride | 9 mg |
| Sodium chloride | 9 mg |
| Sodium hydroxide | 3 mg |
| | (approximately) |
| Water for injection | 1 mL |

Quantity of sodium hydroxide is an approximation as it is used for pH adjustment

Sterilization is achieved by filtering the bulk drug product through two (2) inline 0.22 μm sterilizing filters, followed by aseptic filling into amber ISO6R Type 1 glass vials. Bulk drug product was historically filled flint ISO6R Type 1 glass vials, and then amber vials. The present inventors observed particulate formation in both the flint glass and amber vials. Once filled, the vials are sealed with a 20 mm silicone stopper and secured with a 20 mm flip off seal. The ISO6R glass vials comply with USP [symbol] 661 [symbol] , Chemical Resistance-Glass Containers. The silicone stoppers comply with USP [symbol] 381 [symbol] -Elastomer Closures for Injections.

SnPP sterile solution is a parenteral drug product that is administered via IV infusion. USP requires injectable drug products to be analyzed for particulate matter unless exempt through use of a final filter.

SnPP can be made using a method according to a traditional 4-step process or 2-step process as described below. These processes and details of the resulting compositions are fully described in U.S. patent application Ser. No. 18/428,728 and PCT/US24/13764, entitled “NOVEL SN-PROTOPORPHYRIN COMPOSITIONS, METHODS OF MAKING, AND USES THEREOF” and filed on Jan. 31, 2024, the contents of which are fully incorporated by reference herein.

### SnPP 4-Step Process

A first composition of Stannic protoporfin was made using a 4-step process as follows.

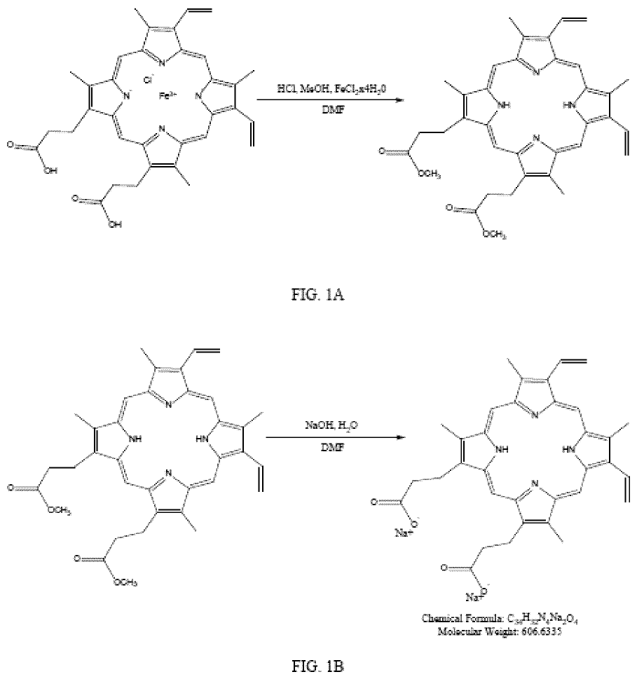

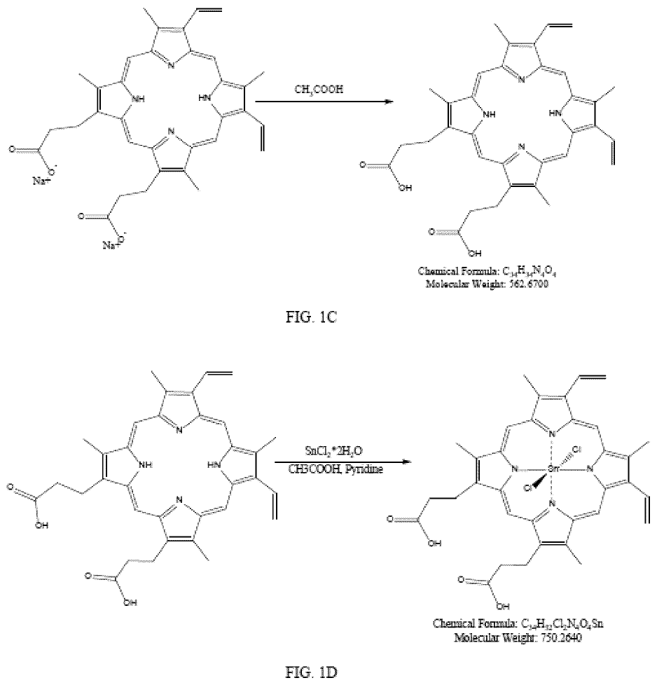

Hemin was dissolved in dimethylformamide at 50-60° C. and filtered. In a separate vessel methanol was saturated with gaseous HCl such that it was heated to 45° C. multiple times. Iron (II) chloride tetrahydrate was then added to the methanol/HCl solution. At this point, the filtered Hemin solution was added by peristaltic pump at ˜25 ml/min such that the temperature was maintained between 40-60° C. Following complete addition of the hemin solution, the reaction was stirred under HCl gas for another 30 minutes. The reaction mixture was then diluted with dichloromethane and washed with water. Aqueous washes are then back-extracted with dichloromethane. The organics were combined and washed once more with water. Following separation, the organics were then split into two portions and purified by silica gel chromatography using a dichloromethane, ethyl acetate gradient. Product fractions were then combined and stripped of solvent by rotary evaporation until ˜25% of the original volume was reached. Ethyl acetate was then added and rotary evaporation was continued and the final slurry was then filtered to isolate a dark purple solid which is protoporphyrin IX dimethyl ester as shown in FIG. 1A.

Protoporphyrin IX dimethyl ester was dissolved in dimethylformamide at 105° C. under inert atmosphere. Aqueous NaOH was added and the temperature was increased to 110° C. The reaction was stirred for 3 hours and allowed to cool. The following day, the reaction was cooled to <6° C. and filtered. The crude protoporphyrin IX disodium salt was washed with cold DMF and acetone. After the product has sufficiently dried on the filter, it was ground with a mortar and pestle and dried in a vacuum over at 80° C. overnight. This step is illustrated in FIG. 1B.

Protoporphyrin IX disodium salt was dissolved in glacial acetic acid under inert atmosphere and stirred at room temperature for at least 24 hours. The reaction was filtered to isolate a red/brown solid. The product was then suspended in 0.5 N acetic acid, stirred overnight and filtered again. Depending on purity of the protoporphyrin IX, one or more recrystallizations was carried out using pyridine at 80° C. followed by cooling to −20° C. and filtration as shown in FIG. 1C.

Glacial acetic acid and pyridine were charged into a 22 L reaction vessel with inert atmosphere at 50° C. Stannous chloride dihydrate was added and stirred to complete dissolution. Protoporphyrin IX was then added and stirred at temperature for a minimum of 48 hours. The resulting solution was then cooled to room temperature, filtered, and rinsed with glacial acetic acid. The final product was then vacuum dried and slurried in hydrochloric acid followed by filtration, deionized water rinse, and finally vacuum drying. This step is shown in FIG. 1D.

### SnPP Two-Step Process

A first composition of Stannic protoporfin was made using a two-step process in accordance with an embodiment of the present invention, as follows:

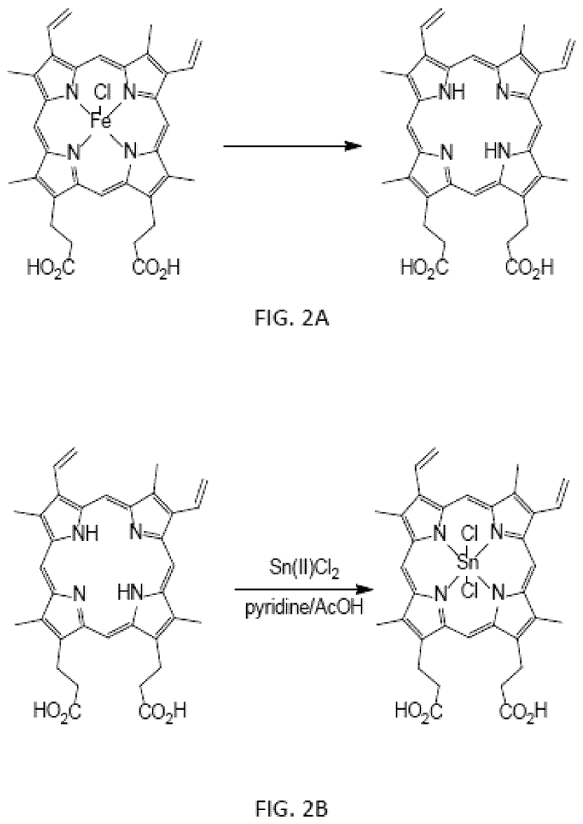

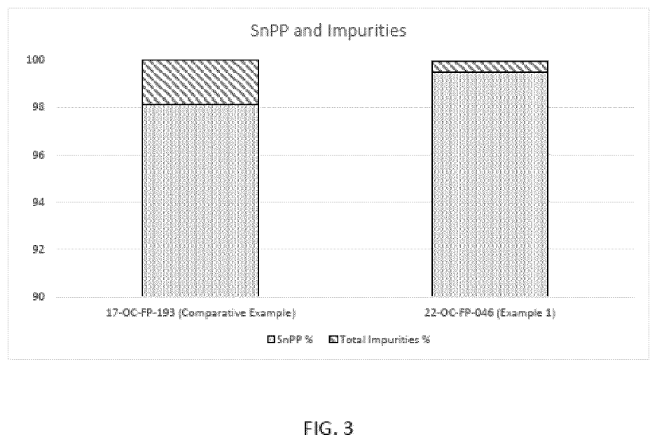

Hemin was dissolved in hot formic acid, then iron powder was added in aliquots over 20 min. The resulting mixture was heated and stirred for 30 min, then filtered through Celite. The filtrate was added to a stirring aqueous solution of NH₄OAc to precipitate the desired product, which was filtered and dried. This crude material was dissolved in hot pyridine and the hot solution was filtered through Celite. The purified product precipitated from the filtrate upon cooing and was recovered by filtration. This corresponds to the first step in this process as shown in FIG. 2A.

Stannous chloride was dissolved in pyridine under inert atmosphere, glacial acetic acid was added and the mixture is heated at 50° C. Protoporphyrin IX was then added and stirred and heated for a minimum of 24 hours and monitored for completion by HPLC. The reaction was cooled to room temperature and filtered. The product was then triturated first with water, then 2 M HCl_{(aq)} and then again with water. An IPC was conducted to determine if a pyridine/AcOH recrystallization, followed by an additional water trituration. The product was then dried to remove residual solvents. This corresponds to the second step in this process as shown in FIG. 2B.

### Particulate Matter Analysis

Particulate matter is analyzed per USP [symbol] 788 [symbol] -Particulate Matter in Injections, using Method I as required, and Method II as needed. As the packaged drug product is a small-volume parenteral (less than 25 mL), the analysis is performed by pooling and mixing the contents of 10 or more vials. Four aliquots (NLT 5 mL each) of the mixture are analyzed using a light obscuration particle counter (HIAC 9703+). The results of the first aliquot are disregarded, and the mean number of particles is evaluated for aliquots two-four, against Method I Test 1.B criteria. The results meet acceptance criteria if the average number of particles present does not exceed 6000 per container ≥10 μm and does not exceed 600 per container ≥25 μm.

If Method I acceptance criteria is not met, the analysis must proceed to Method II, using a binocular microscope and suitable membrane filter. The pooled product (the same sample pool may be used if volume is adequate) is vacuum filtered through a 1.0 μm or finer membrane filter, followed by air drying the filer in a petri dish. The filter is then evaluated under microscope at 100±10 magnifications against Method II Test 2.B criteria. The results meet acceptance criteria if the average number of particles present does not exceed 3000 per container ≥10 μm and does not exceed 300 per container ≥25 μm.

Stannic protoporfin injection contains 9 mg/ml of stannic protoporfin and undergoes sterile filtration into 5 mL sterile vials. The vials are crystal zenith cyclic olefin polymer which are designed for high pH solutions and which meet the exterior dimensions of ISO glass standard 8362-1. The vials meet the requirements of USP <661.1> Plastic Materials of Construction. Once filled, the vials are sealed with a 20 mm silicone stopper and secured with a 20 mm flip off seal. The silicone stoppers comply with USP [symbol] 381 [symbol] -Elastomer Closures for Injections. The specification for the stopper can be found in Table 3.2.P.7-2 and the specification for the seal can be found in Table 3.2.P.7-3.

TABLE 3.2.P.7-1
Specifications for Crystal Zenith Vials

| Test | Acceptance Criterion |
|---|---|
| Inner Diameter | 12.30-12.70 mm |
| Outer Diameter | 19.50-19.90 mm |
| Overall Height | 38.00-39.00 mm |
| Absorbance | NMT 0.2 |
| Acidity or Alkalinity | NaOH: NMT 1.5 mL |
| | HCl: NMT 1 mL |
| Total Organic Carbon | NMT 5 mg/L |
| Phenolic Antioxidants | NMT 0.3% |
| Bacterial Endotoxin USP | NMT 0.25 EU/mL |
| <85> | |
| Sterility USP <71> | No Growth |
| Particulate Matter | <6000 particles ≥ 10 μm |
| USP<788> | <600 particles ≥ 25 μm |

NMT = not more than

The crystal zenith vials are manufactured by Daikyo Seiko, Ltd. in Sano, Japan. All CZ molding processes take place in ISO 7 (Class 10,000) environments. The products are 100 percent inspected to ensure product quality attributes are met. Water or aqueous-based substances do not come into contact with the molded vials; therefore, the product is tested and certified as non-pyrogenic.

### Particulate Matter Analysis for Glass Vials

SnPP was prepared according to the 2-step process noted above, and filled in Crystal Zenith and Type I Borosilicate glass, respectively. The drug product batches were analyzed for particulate matter at release, and annually at accelerated (40±2° C./75±5% RH) and long term (25±2° C./60±5% RH) stability conditions.

| Timeframe | Condition | Crystal Zenith | Type I Borosilicate |
|---|---|---|---|
| Initial | N/A | 904 particles ≥ 10 μm | 3,752 particles ≥ 10 μm |
| | | <1 particles ≥ 25 μm | 23 particles ≥ 25 μm |
| 1 month | 25° C. / 60% RH | 1,744 particles ≥ 10 μm | N/A |
| | | 10 particles ≥ 25 μm | |
| | 40° C. / 75% RH | 782 particles ≥ 10 μm | 31,967 particles ≥ 10 μm |
| | | 65 particles ≥ 25 μm | 157 particles ≥ 25 μm |
| 2 month | 25° C. / 60% RH | 1,427 particles ≥ 10 μm | N/A |
| | | 5 particles ≥ 25 μm | |
| | 40° C. / 75% RH | 329 particles ≥ 10 μm | |
| | | 4 particles ≥ 25 μm | |
| 3 month | 25° C. / 60% RH | 1,184 particles ≥ 10 μm | 32,459 particles ≥ 10 μm |
| | | 2 particles ≥ 25 μm | 184 particles ≥ 25 μm |
| | 40° C. / 75% RH | 184 particles ≥ 10 μm | 32,131 particles ≥ 10 μm |
| | | 1 particles ≥ 25 μm | 82 particles ≥ 25 μm |
| 4 month | 25° C. / 60% RH | 545 particles ≥ 10 μm | N/A |
| | | 1 particles ≥ 25 μm | |
| | 40° C. / 75% RH | 440 particles ≥ 10 μm | |
| | | 0 particles ≥ 25 μm | |
| 6 month | 25° C. / 60% RH | 1,038 particles ≥ 10 μm | 33,720 particles ≥ 10 μm |
| | | 4 particles ≥ 25 μm | 167 particles ≥ 25 μm |
| | 40° C. / 75% RH | 2,021 particles ≥ 10 μm | 23,807 particles ≥ 10 μm |
| | | 2 particles ≥ 25 μm | 60 particles ≥ 25 μm |
| 9 month | 25° C. / 60% RH | 3,316 particles ≥ 10 μm | N/A |
| | | 1 particles ≥ 25 μm | |
| | 40° C. / 75% RH | 830 particles ≥ 10 μm | |
| | | 4 particles ≥ 25 μm | |
| 12 month | 25° C. / 60% RH | 453 particles ≥ 10 μm | 29,324particles ≥ 10 μm |
| | | 1 particles ≥ 25 μm | 81 particles ≥ 25 μm |
| | 40° C. / 75% RH | N/A | N/A |

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

1. A stable stannic protoporfin product, comprising:
a cyclic olefin polymer container;
an aqueous solution within the container comprising:
stannic protoporfin;
water for injection, wherein the aqueous solution has a pH in the range of 11.0 to 13.0; and
a silicone stopper enclosing the aqueous solution within the cyclic olefin polymer container.

2. The stable stannic protoporfin product of claim 1, wherein the aqueous solution comprises added sodium hydroxide and sodium chloride.

3. The stable stannic protoporfin product of claim 1, wherein the stable stannic protoporfin product exhibits <4,000 particles of size ≥10 μm over a period of 12 months at 25° C./60% RH.

4. The stable stannic protoporfin product of claim 3, wherein the stable stannic protoporfin product exhibits <20 particles of size ≥25 μm over a period of 12 months at 25° C./60% RH.

5. The stable stannic protoporfin product of claim 1, wherein the stable stannic protoporfin product exhibits <2,500 particles of size ≥10 μm over a period of 9 months at 40° C./75% RH.

6. The stable stannic protoporfin product of claim 5, wherein the stable stannic protoporfin product exhibits <80 particles of size ≥25 μm over a period of 9 months at 40° C./75% RH.

7. The stable stannic protoporfin product of claim 1, wherein the product includes a light blocking material on the outside of the cyclic olefin polymer container that reduces exposure of the aqueous solution to light.

8. A method of administering stannic protoporfin comprising removing the stable stannic protoporfin product of claim 1 from an opaque container, and administering the stannic protoporfin to a patient in need thereof.

9. The method of claim 8, wherein the stannic protoporfin is combined with a second aqueous composition comprising iron sucrose to form a third aqueous composition, and the administration comprises intravenously infusing the third aqueous composition to the patient within 24 to 48 hours before a scheduled surgery.

10. The method of claim 9, wherein the surgery is one or more of coronary artery bypass graft (CABG), cardiac valve, or combined CABG/valve surgery on cardiopulmonary bypass (CPB).

11. The method of claim 9, wherein the surgery is an organ transplant surgery.

12. The method of claim 9, wherein the third aqueous composition comprises stannic protoporfin, iron sucrose, and bicarb.

13. The stable stannic protoporfin product or method of of claim 1, wherein the stannic protoporfin composition comprises a compound of Formula (I) 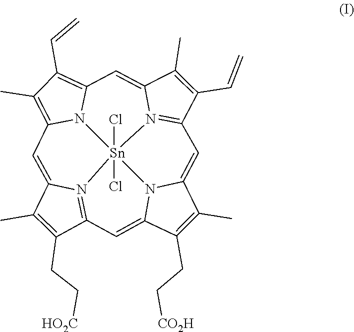
wherein the composition comprises a level of mesoporphyrin of Formula (III) 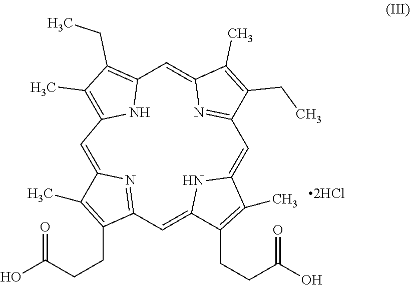 [SMILES: CCC1=C(C)c2cc3[nH]c(cc4nc(cc5[nH]c(cc1n2)c(C)c5CCC(=O)O)C(CCC(=O)O)=C4C)c(C)c3CC]
that is less than 1.0 wt. % and a total impurity level below 3 wt. %.

14. The method of claim 8, wherein the stannic protoporfin composition comprises a compound of Formula (I) 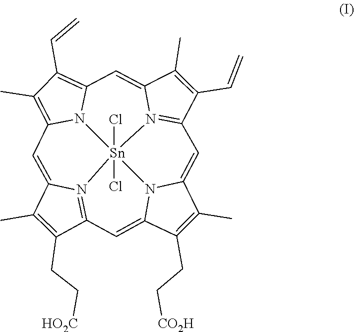
wherein the composition comprises a level of mesoporphyrin of Formula (III) 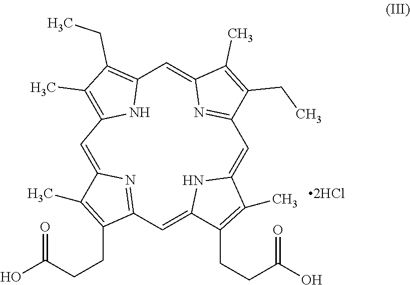 [SMILES: CCC1=C(C)c2cc3[nH]c(cc4nc(cc5[nH]c(cc1n2)c(C)c5CCC(=O)O)C(CCC(=O)O)=C4C)c(C)c3CC]
that is less than 1.0 wt. % and a total impurity level below 3 wt. %.
